(19) United States
(12) Patent Application Publication
(10) Pub. No.: US 2008/0093218 A1
(43) Pub. Date: Apr. 24, 2008

(54) Method for screening thermal stable proteins

(75) Inventors: Shir-Ly Huang, Taoyuan (TW); Zhang-yan Huang, Zhonghe City (TW)

Correspondence Address: TROXELL LAW OFFICE PLLC, SUITE 1404, 5205 LEESBURG PIKE, FALLS CHURCH, VA, 22041, US

(73) Assignee: National Central University, Taoyuan (TW)

(21) Appl. No.: 11/790,830

(22) Filed: Apr. 27, 2007

(30) Foreign Application Priority Data
Oct. 23, 2006 (TW) 095139059

(51) Int. Cl. G01N 27/26; G01N 33/68

(52) U.S. Cl. 204/450; 436/86

(57) ABSTRACT

The present invention screens a number of thermal stable proteins at a time. And the proteins are rapidly identified by using a mass spectrometry. The proteins obtained after screening can be gene cloned to determine its soluble temperature. By using the present invention, time, cost and human resource can be saved.

### FIELD OF THE INVENTION

The present invention relates to screen proteins; more particularly, relates to processing a heat treatment at different temperatures and using a mass spectrometry to rapidly determine thermal stable proteins.

### DESCRIPTION OF THE RELATED ARTS

Thermal stability data of a protein are essential to utilize and produce a biochemical and commercially importing substance. Modern studies on thermal stability of protein is done by comparing homologous proteins to find out significant characteristics of the thermal stability; and it is found that specific amino-acids and their distributions on a protein sequence, a hydrogen bond, a disulfide bond and a salt bridge of protein structure play critical roles to the thermal stability. Hence, screening out a great amount of proteins is almost a mission impossible especially for those thermal stable proteins whose genes have unrecognized functions or sequences can not be found.

On the other hand, a living temperature for an organism is an important parameter for studying the thermal stability of a protein, which is expressed as Tₘ=24.4+0.93 Tₑₙᵥ. Therein, Tₘ means a melting temperature of the protein and Tₑₙᵥ means an environmental temperature of the origin organism. As a result, on studying thermal stable proteins, a thermal stable temperature can be only obtained by the most suitable living temperature for the organism, though it is not the exact thermal stable temperature. Hence, the prior arts do not fulfill users' requests on actual use.

### SUMMARY OF THE INVENTION

The main purpose of the present invention is to rapidly screen thermal stable proteins with a mass spectrometry.

To achieve the above purpose, the present invention is a method for screening thermal stable proteins, where, at first, a proteome from an organism is processed through a heat treatment; a process through an ultracentrifugation is followed to obtain a supernatant as a soluble proteome; and, at last, a high throughput analysis is processed with a mass spectrometry to rapidly determine protein identifications. Accordingly, a novel method for screening thermal stable proteins is obtained.

### BRIEF DESCRIPTIONS OF THE DRAWINGS

The present invention will be better understood from the following detailed description of the preferred embodiment according to the present invention, taken in con junction with the accompanying drawings, in which

FIG. 1 is the flow view showing the preferred embodiment according to the present invention; and

FIG. 2 is the view showing the curve of Geobacillus kaustophilus ATCC8005 after being treated at various temperature.

### DESCRIPTION OF THE PREFERRED EMBODIMENT

The following description of the preferred embodiment is provided to understand the features and the structures of the present invention.

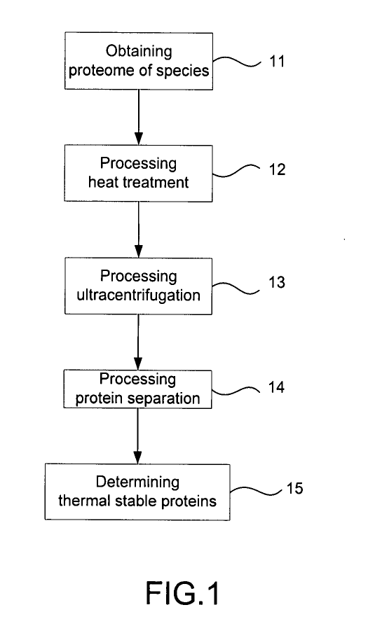

Please refer to FIG. 1 which is a flow view showing a preferred embodiment according to the present invention. As shown in the figure, the present invention is a method for screening thermal stable proteins, comprising the following steps:

(a) Obtaining a proteome of a species cell 11: A proteome of a species is obtained. The species can be a microorganism, a plant or an animal; and the proteome can be obtained from cytoplasmic proteins, organelle proteins or secretory proteins.

(b) Processing a heat treatment 12: The proteome is processed through a heat treatment.

(c) Processing an ultracentrifugation 13: Soluble proteins are obtained by processing the proteome through an ultracentrifugation, where the proteins obtained after the heat treatment are not precipitable.

(d) Processing a protein separation 14: The soluble proteins are processed through a protein separation by a purification method, such as electrophoresis or liquid chromatography, to be separated.

(e) Determining thermal stable proteins 15: Trypsin is used to digest the separated proteins; and the peptide mixture of the soluble proteins are obtained. Peptide mass fingerprints or peptide sequences are obtained through a high throughput analysis to rapidly determine thermal stable proteins by using a matrix-assisted laser desorption/ionization time-of-flight mass spectrometry (MALDI-Q-TOFMS), a surface-assisted laser desorption/ionization time-of-flight mass spectrometry (SELDI-Q-TOFMS), or an electrospray ionization quadruple time-of-flight mass spectrometer (ESI-Q-TOFMS).

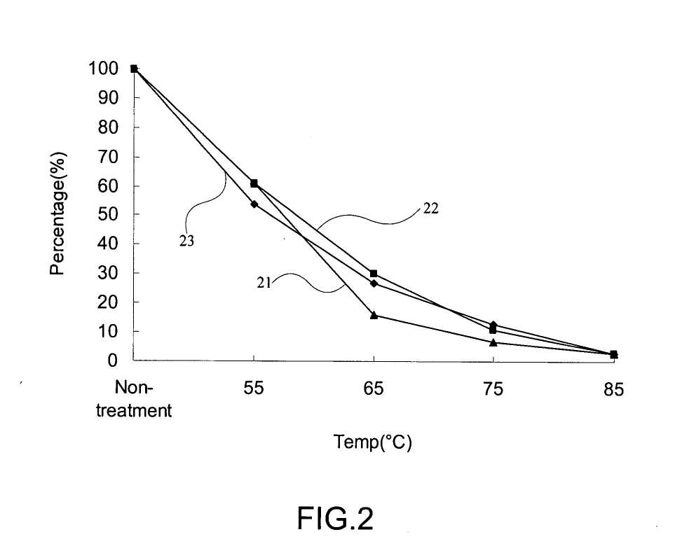

Please refer to FIG. 2, which is a view showing a curve of. Geobacillus kaustophilus ATCC8005 after being treated at various temperature. As shown in the figure, the present invention is applied to a species, Geobacillus kaustophilus ATCC8005, to obtain changes in quality of a soluble proteome of the species cell. The initial protein concentrations for the proteome are set at 1.2 milligrams per milliliter (mg/ml), 6 mg/ml and 12 mg/ml. Then the proteome is processed for 30 minutes under 55 Celsius degrees (° C.), 65° C., 75° C. or 85° C. As shown in the figure, a percent of total protein remained in a heat-stable sub-proteome does not differ, even when having a various initial protein concentration.

The proteins obtained from the Geobacillus kaustophilus ATCC8005 after the heat treatment and the ultracentrifugation are processed through electrophoresis with a 12˜20% polyacrylamide gel. After being treated under 85° C. (85° C.-treatment) for 30 minutes, some proteins are still soluble, where there are 32 proteins identified. The identified proteins are then processed with trypsin and a mass spectrometry is used to rapidly and accurately determine thermal stable proteins under 85° C. At this moment, a quality mark of a standard protein is obtained as a reference for the molecular weights of the proteins obtained after being determined by the mass spectrometry; thus, accuracy is testified.

To sum up, the present invention is a method for screening thermal stable proteins, where a proteome of a species is processed through heat treatments under various temperatures; the proteome is then processed through an ultracentrifugation to obtain soluble proteins which are not precipitable after the heat treatment; after the soluble proteins are separated and the initial protein is digested by trypsin, PMFs or peptide sequences are processed through a high throughput analysis with a mass spectrometry to rapidly determine thermal stable proteins.

The preferred embodiment herein disclosed is not intended to unnecessarily limit the scope of the invention. Therefore, simple modifications or variations belonging to the equivalent of the scope of the claims and the instructions disclosed herein for a patent are all within the scope of the present invention.

What is claimed is:

1. A method for screening thermal stable proteins, comprising steps of:
(a) obtaining a proteome of a species;
(b) processing said soluble proteome through a heat treatment;
(c) processing said soluble proteome through an ultracentrifugation to obtain soluble proteins;
(d) processing said soluble proteins through a protein separation to obtain a peptide mixture;
(e) processing said peptide mixture through a high throughput analysis with a mass spectrometry to obtain a result selected from a group consisting of a peptide sequence or a peptide mass finger print to identify thermal stable proteins.

2. The method according to claim 1,
wherein said species is selected from a group consisting of a microorganism, a plant and an animal.

3. The method according to claim 1,
wherein said proteome is obtained from proteins selected from a group consisting of cytoplasm, organelle, a secretory protein and a body fluid.

4. The method according to claim 1,
wherein said soluble proteins obtained after said heat treatment in step (c) is not precipitable.

5. The method according to claim 1,
wherein said protein separation is obtained through a purification method selected from a group consisting of electrophoresis and liquid chromatography.

6. The method according to claim 1,
wherein said mass spectrometry used in step (e) is a matrix-assisted laser desorption/ionization time-of-flight mass spectrometry (MALDI-Q-TOFMS).

7. The method according to claim 1,
wherein said mass spectrometry used in step (e) is a surface-assisted laser desorption/ionization time-of-flight mass spectrometry (SELDI-Q-TOFMS).

8. The method according to claim 1,
wherein said mass spectrometry used in step (e) is an electrospray ionization quadruple time-of-flight mass spectrometer (ESI-Q-TOFMS).
